(19) United States
(12) Patent Application Publication
(10) Pub. No.: US 2019/0001159 A1
(43) Pub. Date: Jan. 3, 2019

(54) STEERABLE HIGH-INTENSITY FOCUSED ULTRASOUND (HIFU) ELEMENTS

(71) Applicants: Kailiang Chen, Branford, CT (US); Lawrence C. West, San Jose, CA (US); Jaime Scott Zahorian, Guilford, CT (US); Nevada J. Sanchez, Guilford, CT (US); Tyler S. Ralston, Clinton, CT (US)

(72) Inventors: Kailiang Chen, Branford, CT (US); Lawrence C. West, San Jose, CA (US); Jaime Scott Zahorian, Guilford, CT (US); Nevada J. Sanchez, Guilford, CT (US); Tyler S. Ralston, Clinton, CT (US)

(73) Assignee: Butterfly Network, Inc., Guilford, CT (US)

(21) Appl. No.: 16/022,799

(22) Filed: Jun. 29, 2018

Related U.S. Application Data
(60) Provisional application No. 62/563,616, filed on Sep. 26, 2017.
(60) Provisional application No. 62/527,534, filed on Jun. 30, 2017.

(51) Int. Cl. A61N 7/00; A61N 7/02

(52) U.S. Cl. CPC A61N 7/00; A61N 7/02; A61B 8/4477; A61N 2007/0078; A61N 2007/0091; A61N 2007/0052

(57) ABSTRACT

Ultrasound devices configured to perform high-intensity focused ultrasound (HIFU) are described. An ultrasound device may include HIFU units configured to emit high acoustic intensities. Multiple ultrasound devices may be disposed on a substrate, which may be configured to be flexed so that the direction of emission of the ultrasound devices can be mechanically controlled. Additionally, or alternatively, the ultrasound beams produced by different ultrasound devices may be electronically oriented by adjusting the phases of the signals with which each element of a device is driven. For example, multiple phased arrays of ultrasound devices may be used to concentrate ultrasound energy into a desired location. In some embodiments, the time at which different ultrasound signals are emitted may be controlled, for example to ensure that the combined signal has at least a desired intensity.

### CROSS-REFERENCE TO RELATED APPLICATIONS

This application claims the benefit under 35 U.S.C. § 119(e) of U.S. Provisional Patent Application Ser. No. 62/527,534, entitled “STEERABLE HIGH-INTENSITY FOCUSED ULTRASOUND (HIFU) ELEMENTS,” filed on Jun. 30, 2017 under Attorney Docket No. B1348.70052US00, which is hereby incorporated herein by reference in its entirety.

This application claims the benefit under 35 U.S.C. § 119(e) of U.S. Provisional Patent Application Ser. No. 62/563,616, entitled “STEERABLE HIGH-INTENSITY FOCUSED ULTRASOUND (HIFU) ELEMENTS,” filed on Sep. 26, 2017 under Attorney Docket No. B1348.70052US01, which is hereby incorporated herein by reference in its entirety.

### TECHNICAL FIELD

The present application relates to systems and techniques for performing high intensity focused ultrasound (HIFU).

### BACKGROUND

High intensity focused ultrasound (HIFU) is used in some medical procedures to kill cancer cells with high frequency sound waves. These waves deliver a strong beam to a specific part of a cancer. Some cells die when this high intensity ultrasound beam is focused directly onto them.

### SUMMARY

Some embodiments relate to an apparatus comprising a substrate having a plurality of support portions including a first support portion and a second support portion, wherein the first support portion is connected to the second support portion through a coupler; a first plurality of ultrasound elements configured as high-intensity focused ultrasound (HIFU) elements and disposed on the first support portion of the substrate; and a second plurality of ultrasound elements configured as HIFU elements and disposed on the second support portion of the substrate.

In some embodiments, the first and second plurality of ultrasound elements comprise capacitive micromachined ultrasound transducers (CMUT).

In some embodiments, the coupler is selected from the group consisting of a hinge, a spring, a flexure, a beam, a joint and a sphere.

In some embodiments, the apparatus further comprises a third plurality of ultrasound elements configured to receive ultrasound signals and disposed on the first support portion of the substrate.

In some embodiments, the third plurality of ultrasound elements are configured as ultrasound imaging elements.

In some embodiments, the apparatus further comprises a servomotor coupled to the substrate.

In some embodiments, at least a portion of the first plurality of ultrasound elements and/or at least a portion of the second plurality of ultrasound elements are configured to receive ultrasound signals.

In some embodiments, the apparatus further comprises an actuator coupled to the substrate, the actuator being configured to move the first support portion relative to the second support portion.

In some embodiments, the actuator is selected from the group consisting of a pneumatic actuator, a hydraulic actuator and a servomotor.

Some embodiments relate to an apparatus comprising a plurality of ultrasound elements disposed on a semiconductor substrate; and a plurality of signal drivers, each of the plurality of signal drivers being coupled to a respective ultrasound element of the plurality of ultrasound elements.

In some embodiments, the plurality of signal drivers are disposed on the semiconductor substrate.

In some embodiments, the plurality of ultrasound elements comprises micromachined ultrasound transducers (CMUT).

In some embodiments, each of the plurality of signal drivers comprises a phase shifter and/or an adjustable delay element.

Some embodiments relate to a high intensity focused ultrasound (HIFU) apparatus, comprising a plurality of HIFU ultrasound-on-a-chip probes configured to emit electronically steerable beams, the plurality of HIFU ultrasound-on-a-chip probes being coupled to a support.

In some embodiments, the HIFU apparatus further comprises a controller coupled to the plurality of HIFU ultrasound-on-a-chip probes and configured to control beam steering of the plurality of HIFU ultrasound-on-a-chip probes by adjusting relative phases of the electronically steerable beams.

In some embodiments, the controller is configured to control beam steering of the plurality of HIFU ultrasound-on-a-chip probes by controlling a direction of emission and/or a focal length.

In some embodiments, a first HIFU ultrasound-on-a-chip probe comprises an arrangement of CMUTs configured to provide HIFU.

In some embodiments, a first HIFU ultrasound-on-a-chip probe of the plurality of HIFU ultrasound-on-a-chip probes comprises an arrangement of capacitive micromachined ultrasound transducers (CMUTs) configured to provide HIFU.

In some embodiments, the support comprises a plurality of support portions, the support portions being mechanically movable relative to each other.

In some embodiments, at least one of the plurality of HIFU ultrasound-on-a-chip probes is configured to emit an acoustic intensity that is between 500 W/cm² and 20 KW/cm².

Some embodiments relate to a method, comprising emitting, using an high-intensity focused ultrasound (HIFU) apparatus, at least one ultrasound signal towards at least one target region; and adjusting, based on the emitting, direction of the at least one ultrasound signal via electronic steering.

In some embodiments, the at least one ultrasound signal is generated using at least one ultrasound element.

In some embodiments, the at least one ultrasound element includes at least one selected from the group consisting of: a micromachined ultrasound transducer (CMUT), piezoelectric transducer, lead zirconate titanate (PZT) element, lead magnesium niobate-lead titanate (PMN-PT) element, polyvinylidene difluoride (PVDF) element, high power ceramic element, and a PZT-4 ceramic element.

In some embodiments, the at least one ultrasound signal includes a high-intensity focused ultrasound (HIFU) signal and/or a non-HIFU ultrasound signal.

In some embodiments, the method further comprises adjusting, based on the emitting, direction of the at least one ultrasound signal via mechanical steering.

In some embodiments, the electronically steering the at least one ultrasound signal includes controlling a phase of the at least one ultrasound signal.

In some embodiments, the mechanically steering the at least one ultrasound signal includes adjusting position coordinates of at least one ultrasound element emitting the at least one ultrasound signal in relation to the at least one target region.

In some embodiments, the electronically steering the at least one ultrasound signal includes controlling a time delay of the at least one ultrasound signal.

In some embodiments, adjusting direction of the at least one ultrasound signal via mechanical steering includes adjusting position coordinates of at least one ultrasound element emitting the at least one ultrasound signal in relation to the at least one target region.

In some embodiments, adjusting direction of the at least one ultrasound signal via electronic steering includes controlling a phase of the at least one ultrasound signal.

In some embodiments, adjusting direction of the at least one ultrasound signal via electronic steering includes controlling a time delay of the at least one ultrasound signal.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1A is a schematic diagram illustrating a medical ultrasound device for use with a patient, according to some non-limiting embodiments.

FIG. 1B is a schematic diagram illustrating a substrate having a plurality of ultrasound devices disposed thereon, according to some non-limiting embodiments.

FIG. 1C is a schematic diagram illustrating support portions of the substrate of FIG. 1B while being flexed relative to one another in the yx-plane, according to some non-limiting embodiments.

FIG. 1D is a schematic diagram illustrating support portions of the substrate of FIG. 1B while being flexed relative to one another in the xz-plane, according to some non-limiting embodiments.

FIG. 1E is a schematic diagram illustrating an ultrasound device having a plurality of ultrasound elements arranged as a two-dimensional array, according to some non-limiting embodiments.

FIG. 1F is a schematic diagram illustrating multiple handheld probes comprising respective ultrasound devices, according to some non-limiting embodiments.

FIG. 1G is a schematic diagram illustrating an ultrasound imaging probe being surrounded by multiple HIFU probes, according to some non-limiting embodiments.

FIG. 2A is a schematic diagram illustrating a plurality of servomotors being used in connection with the substrate of FIG. 1B, according to some non-limiting embodiments.

FIG. 2B is a schematic diagram of the substrate of FIG. 1B arranged to conform to the shape of a surface, according to some non-limiting embodiments.

FIG. 2C is a schematic diagram of a system including bladders serving as pneumatic actuators, according to some non-limiting embodiments.

FIG. 2D is a schematic diagram of a system including hydraulic actuators, according to some non-limiting embodiments.

FIG. 2E is a schematic diagram of a system for driving the hydraulic actuators of FIG. 2D, according to some non-limiting embodiments.

FIG. 2F is a schematic diagram of a hydraulic actuator including a spring loaded arm, according to some non-limiting embodiments.

FIG. 3A is a schematic diagram illustrating an ultrasound device comprising a phased array, according to some non-limiting embodiments.

FIGS. 3B, 3D and 3F are plots illustrating respective examples of phases produced by the elements of the phased array of FIG. 3A, according to some non-limiting embodiments.

FIGS. 3C, 3E and 3G are schematic diagrams illustrating examples of acoustic beams produced by the phased array of FIG. 3A, according to some non-limiting embodiments.

FIGS. 3H-3J are plots illustrating respective examples of time delays produced by the elements of the phased array of FIG. 3A, according to some non-limiting embodiments.

FIG. 3K is a schematic diagram illustrating the acoustic beam produced by the phased array of FIG. 3A at different times, according to some non-limiting embodiments.

FIG. 3L is a flowchart illustrating an example of a calibration procedure, according to some non-limiting embodiments.

FIG. 4 is a schematic diagram illustrating multiple ultrasound devices being used to concentrate ultrasound energy in a target region, according to some non-limiting embodiments.

FIG. 5 is a photograph illustrating a non-limiting example of a system for performing high intensity focused ultrasound (HIFU) having a plurality of probes, according to some non-limiting embodiments.

### DETAILED DESCRIPTION

According to an aspect of the present application, mechanically and electronically steerable high intensity focused ultrasound (HIFU) arrangements are provided. The term “steerable” can be used herein to indicate tuning of the direction of emission of an ultrasound signal and/or tuning of the focal length of an ultrasound signal. A HIFU arrangement may include multiple HIFU sources, such as multiple HIFU probes. The probes may be stand-alone separate probes or may be coupled together via an adjustable mechanical coupler. The adjustable mechanical coupler may be configured to adjust in one or more dimensions, allowing for repositioning and/or reorienting of the multiple HIFU sources relative to each other. In some embodiments, the mechanical coupler may be adjustable to accommodate a wide range of anatomical features. The mechanical coupler may be adjusted to facilitate focusing of the HIFU beams from the HIFU sources on a target, such as a common point or points. In some embodiments, the HIFU sources themselves are electronically steerable. For example, the HIFU sources may be ultrasound-on-a-chip devices electronically controllable to perform beam steering. Thus, in some embodiments, a HIFU arrangement is both mechanically and electronically steerable to focus HIFU energy on a static or moving target.

Applicant has appreciated that the ability to concentrate ultrasound energy in a desired region of a human body to perform therapeutic treatments based on high-intensity focused ultrasound (HIFU) can be enhanced by using steerable ultrasound devices. HIFU is a therapeutic technology in which focused ultrasound energy is used to generate highly localized heating to treat human tissues, cancers, cataracts, kidney stones, or other diseases. To generate intensities sufficiently large to produce significant temperature changes, multiple ultrasound beams can be used. However, the regions being targeted are often deep and not easily accessible, thus making it challenging to focus multiple beams on the same location.

In steerable ultrasound devices of the types described herein, the emitted ultrasound beams can be controlled to be directed in a desired direction. According to one aspect of the present application, multiple steerable ultrasound devices may be steered to focus the beams on the same target region, thus increasing the intensity of the resulting wave. Ultrasound beam steering may be achieved in any of numerous ways, including for example via mechanical or electrical means.

In mechanical steering, multiple ultrasound devices may be disposed on a substrate, which may be configured to be flexed so that the direction of emission of the ultrasound devices can be controlled. In some embodiments, a substrate includes multiple support portions coupled to one another via couplers, such as hinges or springs. The couplers may allow the support portions to be flexed relative to one another, thus allowing the ultrasound devices disposed thereon to be directed as desired.

In electrical or electronic steering, the ultrasound beams produced by different devices may be oriented by adjusting the phases of the signals with which each element of a device is driven. In some embodiments, multiple phased arrays of ultrasound devices may be used to concentrate ultrasound energy into a desired location. In some embodiments, the time at which different ultrasound signals are emitted may be controlled, for example to ensure that the combined signal has at least a desired intensity.

The aspects and embodiments described above, as well as additional aspects and embodiments, are described further below. These aspects and/or embodiments may be used individually, all together, or in any combination of two or more, as the application is not limited in this respect.

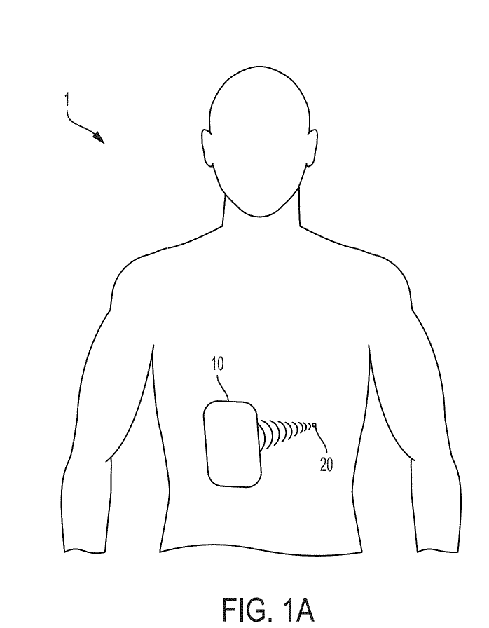

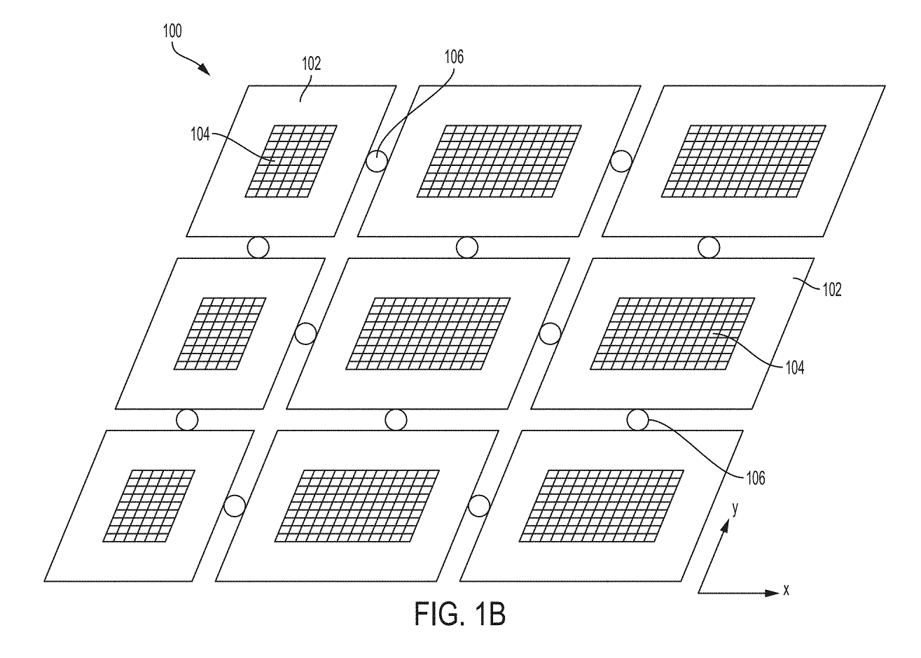

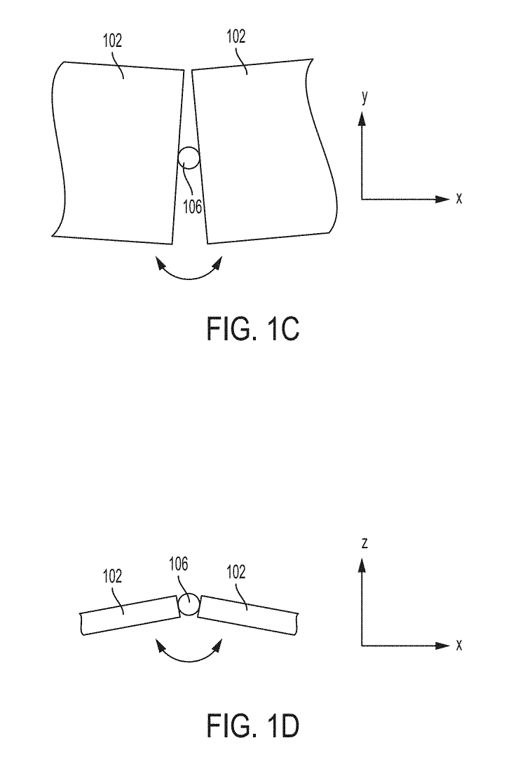

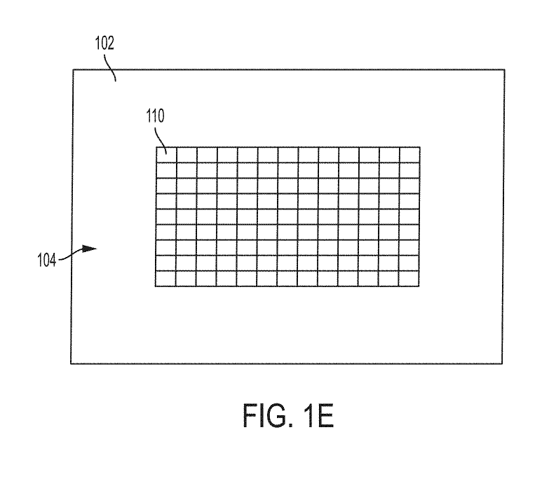

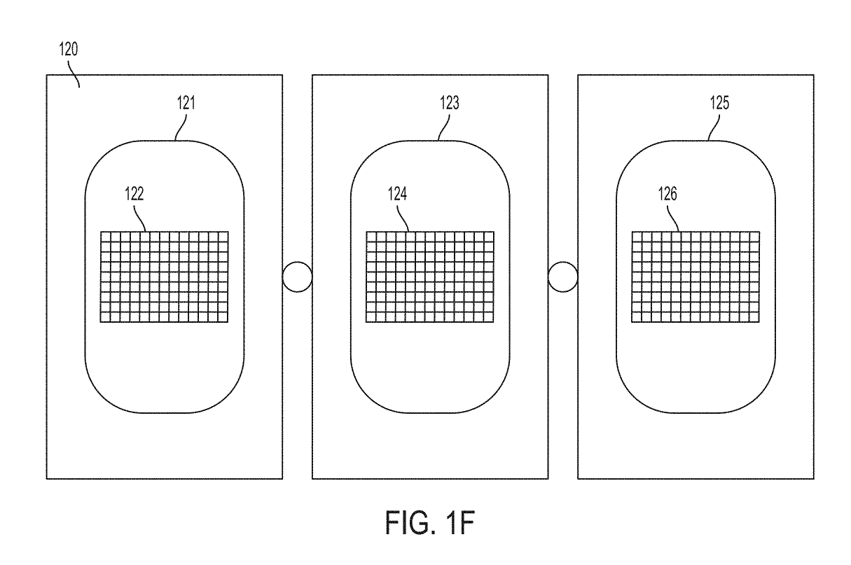

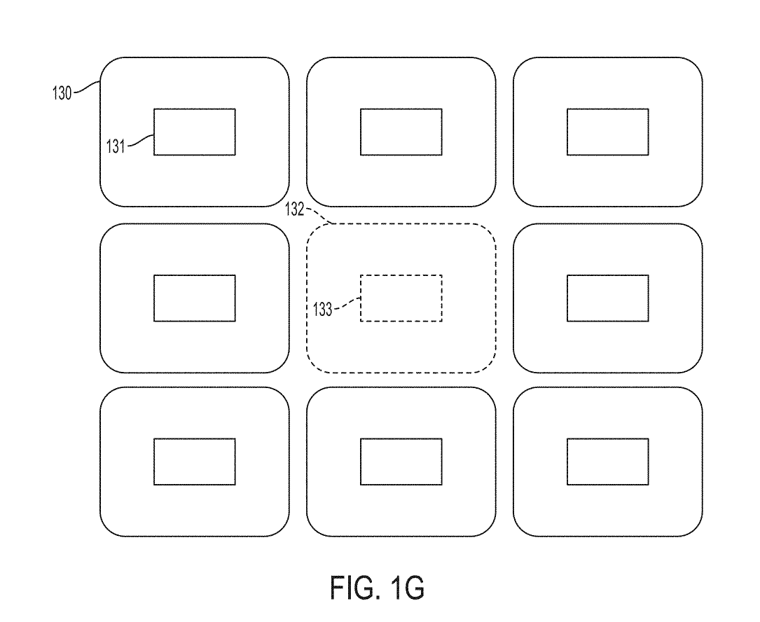

FIG. 1A illustrates a medical ultrasound device 10 for use with a patient 1. Medical ultrasound device 10 may be used for treating medical conditions and/or for performing diagnoses. Medical ultrasound device 10 may include ultrasound elements arranged to provide HIFU and/or ultrasound elements arranged to receive ultrasound signals (for example to perform ultrasound imaging). Medical ultrasound device 10 may be implemented as a handheld probe or as a plurality of handheld probes. In the non-limiting example of FIG. 1A, medical ultrasound device 10 is used to focus HIFU at a target location 20 of patient 1. The target location may represent, for example, a region in need of treatment.

Medical ultrasound device 10 may be implemented in any of numerous ways. In some embodiments, for example, medical ultrasound device 10 may comprise a substrate having a plurality of ultrasound devices. FIG. 1B illustrates schematically a substrate 100 having a plurality of ultrasound devices 104 disposed thereon. Substrate 100 may be made of any of numerous materials, including but not limited to polymers, plastics, metals, semiconductors, or any suitable combination thereof. Substrate 100 may be configured to flex in one, two, or three dimension. In some embodiments, substrate 100 may comprise a plurality of support portions 102 that are interconnected via couplers 106. A coupler 106 may allow the support portions to which it is connected to flex relative to one another. The support portions may be allowed to flex relative to one another in one, two, or three dimensions. For example FIG. 1C and FIG. 1D illustrate two support portions 102 while being flexed relative to one another in the xy-plane and in the xz-plane, respectively. Couplers 106 may be implemented as hinges, springs, flexures, beams, joints, spheres, or other movable mechanisms, and/or any suitable combination thereof.

The support portions 102 may have any suitable shape and size. For example, in some embodiments, at least some support portions 102 may have a shape (viewed in the xy-plane) that is square, rectangular, polygonal, circular, elliptical, or irregular. Of course, not all support portions 102 are limited to a specific arrangement, as different support portions 102 may have different shapes or sizes. While FIG. 1B illustrates a substrate 100 having nine support portions 102, substrates of the types described herein are not limited to any particular number of support portions.

Each ultrasound device 104 may comprise a plurality of ultrasound elements adapted to emit and/or receive ultrasound waves. As such, each ultrasound element may operate as a source and/or a sensor. In some embodiments, these elements may be arranged as two-dimensional arrays (see for example ultrasound elements 110 in FIG. 1E). However, not all ultrasound devices 104 are limited in this respect as some ultrasound elements may be arranged sparsely or irregularly.

Non-limiting examples of ultrasound elements which may be used in any of the embodiments described herein include capacitive micromachined ultrasound transducers (CMUT), piezoelectric transducers, lead zirconate titanate (PZT) elements, lead magnesium niobate-lead titanate (PMN-PT) elements, polyvinylidene difluoride (PVDF) elements, high power (“hard”) ceramics such as those designated as PZT-4 ceramics, or any other suitable elements. Materials designated as PZT-8 materials may be preferable for use as HIFU elements in some embodiments. In some embodiments, ultrasound elements configured as sources may be of a first type while ultrasound elements configured as sensors may be of a second type. By way of a non-limiting example, according to an embodiment, PZT elements may be used to form an array of ultrasound elements configured as sources, while PVDF elements may be used to form an array of ultrasound elements configured as sensors. Such a configuration may be implemented for any purpose(s). In some embodiments, PVDF elements may be more efficient in terms of receiving signals, but may be characterized by an undefined output impedance. Thus, it may be desirable to couple such PVDF elements to high impedance low noise amplifiers (LNAs), which may be best suited for receipt of ultrasound signals rather than sourcing ultrasound signals. PZT elements, on the other hand, may be better suited in some embodiments to operate as ultrasound sources. Thus, embodiments of the present application provide for suitable mixing of radiation element types as sources and sensors to provide desired operation.

In at least some of the embodiments in which the ultrasound elements are implemented using CMUTs, the CMUTs of an ultrasound device may be disposed on a common semiconductor substrate, such as a silicon substrate.

At least some of the ultrasound elements 104 may be configured to operate as high-intensity focused ultrasound (HIFU) elements in some embodiments. In some embodiments, some ultrasound elements may be configured to operate as HIFU elements, while other ultrasound elements may be configured to operate as ultrasound imagers (e.g., for B-mode imaging). In this manner, a single apparatus may perform both HIFU and ultrasound imaging, and therefore may be considered a dual- or multi-modal apparatus. These two types of ultrasound elements may be provided in a common support portion 102 in some embodiments, though not all support portions 102 need to include both types of ultrasound elements. In one example, the ultrasound elements configured as HIFU elements may be interspersed (placed at intervals) among the ultrasound elements configured as imaging elements.

In some of the embodiments including both ultrasound imaging elements and HIFU elements, one or more of the imaging and HIFU elements may be the same as each other. However, in alternative embodiments, the two types of elements may differ. For example, the center frequency, bandwidth, size and/or power specifications may differ for the ultrasound elements configured as imaging elements as compared to those configured as HIFU elements. The types of waveforms transmitted may also differ between the different types of elements. In some embodiments, the ultrasound elements configured as imaging elements may be coupled to different types of circuitry than those configured as HIFU elements.

In some embodiments, the HIFU elements may be configured to emit intensities that are sufficiently large to treat medical conditions (for example through ablation). In some embodiments, the HIFU elements may be configured to emit intensities that are between 500 W/cm² and 20 KW/cm², between 1 KW/cm² and 20 KW/cm², between 1 KW/cm² and 10 KW/cm², between 1 KW/cm² and 9 KW/cm², between 1 KW/cm² and 7 KW/cm², between 1 KW/cm² and 5 KW/cm², between 1 KW/cm² and 3 KW/cm², between 3 KW/cm² and 10 KW/cm², or within any range within such ranges.

For comparison, the intensities emitted by the ultrasound imaging elements may be between 100 mW/cm² and 100 W/cm², between 500 mW/cm² and 100 W/cm², between 1 W/cm² and 100 W/cm², or within any range within such ranges.

HIFU elements, as used herein, are ultrasound elements which may be used to induce a temperature and/or mechanical change in a tissue or a cell. The temperature change may be up to approximately 30 degrees Celsius or more, and may be sufficient in some embodiments to cauterize tissue. However, HIFU elements need not achieve cauterization. For example, less energy than that required for cauterization may be applied. In some embodiments, HIFU elements may be used to achieve heat shock or cause apoptosis (programmed cell death). Achieving such results typically requires less energy than that required to achieve cauterization, but may still be useful in some embodiments. Typically, HIFU elements deposit more power in a subject than conventional ultrasound imaging elements.

In low-temperature thermal HIFU procedures, temperature increases of 5° C. or less may be induced on a tissue for extended periods of time (for example up to three minutes, up to four minutes or up to five minutes) to ensure that cancerous cells are killed without affecting healthy cells. In some such low-temperature procedures, ultrasound beams of 10 mm in diameter at the target plane or less may be applied. In these cases, relatively low HIFU powers, such as less than 10 W or less than 5 W, may be sufficient to cause the desired temperature increases. Low HIFU power may also be used in histotripsy, though the peak intensity may be as high as a 1 KW/cm2 or more.

Alternatively, or additionally, HIFU elements may be used to cause a change in a mechanical property of a tissue or cell. For example, when used in micro-cavitation, HIFU may induce a shock wave at the target location (e.g., at the focal plane of the HIFU). Micro-cavitation may be enabled by applying short HIFU pulses (e.g., between 1 μs and 10 μs) to cause waves of large pressures (e.g., between 5 MPa and 80 MPa). In some embodiments, when short HIFU pulses are applied to a tissue, a vapor cavity or a liquid-free zone (e.g., a bubble) may be formed. A shock wave may be generated when the vapor cavity or liquid-free zone implodes. In some embodiments, bubbles may be formed such that the target region is between bubbles. In some embodiments, the bubbles exhibit high reflectance, which may induce multiple scattering and thus multipath absorption in the tissue.

Alternatively, or additionally, HIFU may be used to perform ablation. To perform ablation, in some embodiments, large temperature increases may be needed, such as up to 57° C. or more. For this reason, large HIFU powers may be used, such as more than 10 W or more than 100 W. To limit diffusion, which may inadvertently damage healthy tissues, short pulses are typically applied, for example with durations of 10 s or less, 5 s or less, or 3 s or less.

Ablation may be performed, at least in some embodiments, once a multipath absorption has been created, for example via micro-cavitation. In this way, the energy needed to perform ablation may be substantially reduced. Furthermore, in this way, the energy outside the target region may be reduced, this limiting damage to healthy tissues located nearby.

According to one aspect of the present application, at least some of the support portions 102 of a substrate 100 may be flexed relative to one another so that the ultrasound waves emitted by the ultrasound devices are concentrated in a desired region, such as a specific tissue of a human body. This may be particularly useful in HIFU applications, in which high intensity is produced by focusing multiple ultrasound waves on the same region. Accordingly, the emitted waves may steered by orienting each support portion 102 according to a particular orientation. The support portions 102 may be oriented manually and/or automatically. In one example, a user may adjust the orientation of the support portions 102, in a trial-and-error fashion, until it is determined that the ultrasound waves are emitted in the desired direction. Different techniques may be used to determine whether the desired location has been hit with sufficiently high intensity. Among these are magnetic resonance imaging and shear wave imaging, in which a change in the elasticity of the tissue is sensed by sensing the velocity (or other characteristics) of a shear wave propagating away from the desired region. Shear waves may be generated by hitting the desired region with an ultrasound wave.

In some embodiments, substrate 100 is mounted in a housing, which may be shaped and sized as a handheld probe. The handheld probe may be operated by a practitioner to perform ultrasound imaging and/or HIFU on a patient. In some embodiments, multiple handheld probes may be used in connection with another, for example to perform HIFU. The handheld probes may comprise respective ultrasound devices arranged as imagers and/or HIFU elements. One example of this configuration is depicted in FIG. 1F, in which handheld probes 121, 123 and 125 are mounted on a support 120. As illustrated, handheld probe 121 may comprise ultrasound device 122, handheld probe 123 may comprise ultrasound device 124, and handheld probe 125 may comprise ultrasound device 126. In some embodiments, the portions of support 120 on which the handheld probes are mounted may be mechanically adjusted relative to one another. For example, couplers of the types described above may be used to allow for each handheld probe to be independently directed. Of course, the arrangement of FIG. 1F may be used in connection with any suitable number of handheld probes.

In one non-limiting example, multiple probes may be arranged such that an ultrasound device configured to perform ultrasound imaging is at least partially surrounded (in two or three dimension) by ultrasound devices configured to perform HIFU. An example of such a configuration is illustrated in FIG. 1G, in which the probes 130 (shown as solid shapes) deliver HIFU and the probe 132 (shown as a dash shape) are arranged to perform ultrasound imaging. As illustrated, probes 130 may include respective ultrasound devices 131 (for delivering HIFU) and probe 132 may include ultrasound device 133 (for performing ultrasound imaging). In some embodiments, while the probe(s) 132 include circuitry for receiving ultrasound waves, some or all the probes 130 may include circuitry for transmitting ultrasound waves without including circuitry for receiving ultrasound waves. In this way, the receiving circuitry may be offloaded to the probe(s) arranged for ultrasound imaging, and as a result, the design of the probes 132 may be simplified. While the configuration of FIG. 1G illustrates the imaging ultrasound device being in the middle of multiple HIFU ultrasound devices, not all embodiments are limited in this respect.

The ultrasound devices of FIG. 1G may be arranged according to any one of the embodiments described herein (such as ultrasound device 104). The beams emitted by ultrasound elements 131 may be steered electronically as described further below. In some embodiments, the probes 130 are mutually coupled via couples 106 (as illustrated in FIG. 1B). In some embodiments, probe 132 is coupled to probes 130 via couplers 106. In some embodiments, probes 130 may be disposed on a substrate having an opening arranged to provide sufficient room to position one or more probes 132 therein.

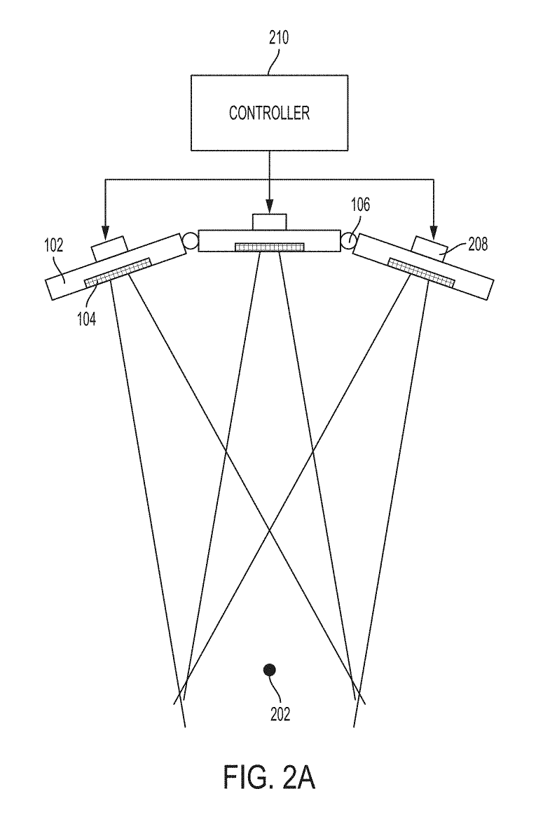

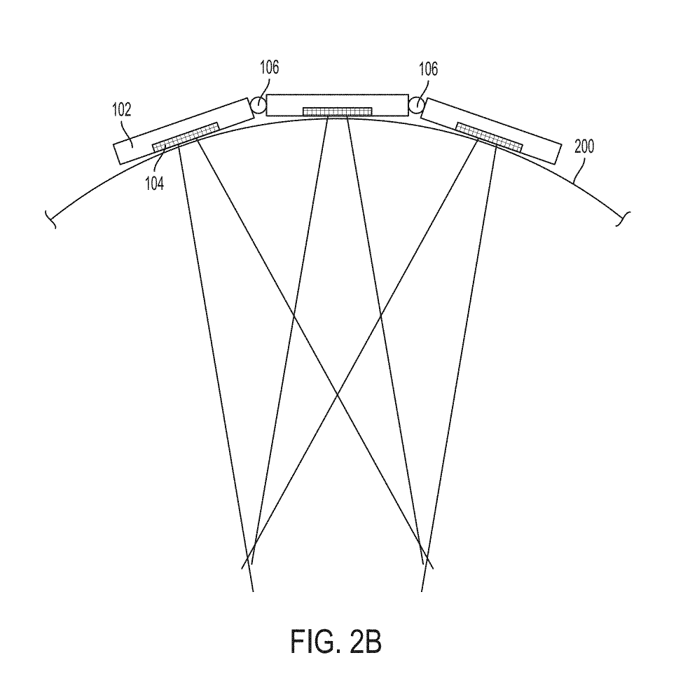

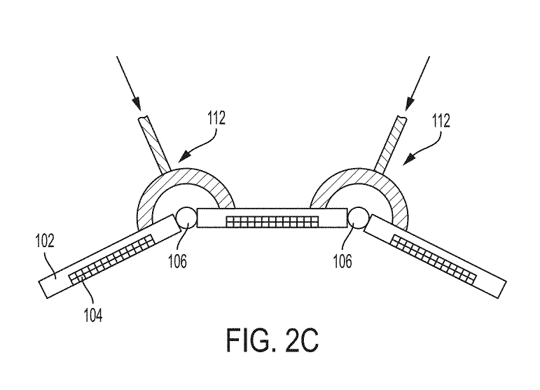

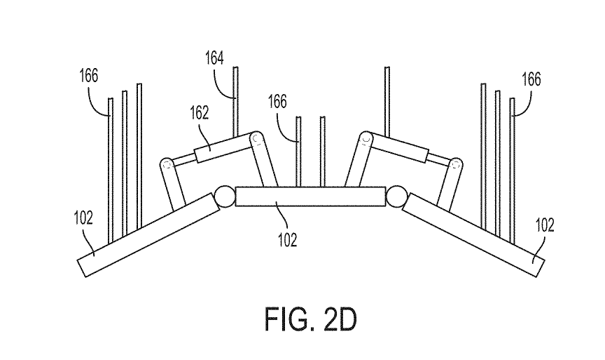

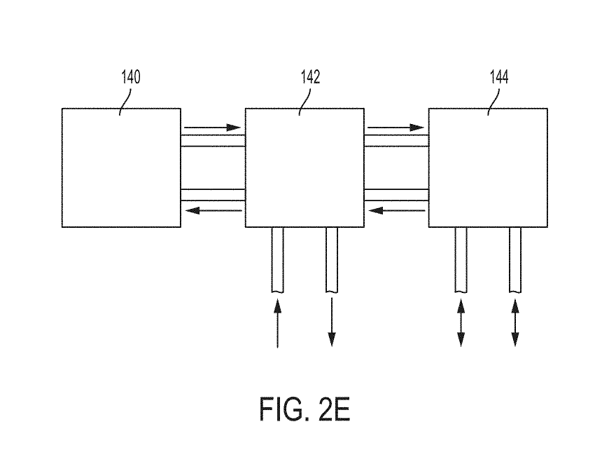

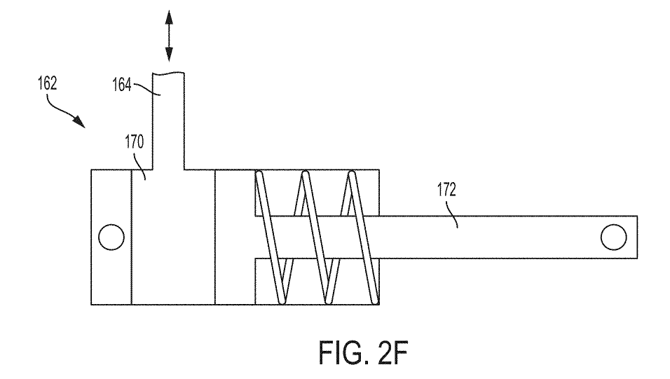

In another example, a substrate 100 may be equipped with one or more servomotors 208 or other servomechanisms, as shown in FIG. 2A. The servomotor(s) or other servomechanisms may flex the substrate by directly actuating support portions 102 and/or couplers 106, and may be controlled using a controller 210 (e.g., a PID controller). In the example of FIG. 2A, the orientation of the support portions 102 is controlled so that the ultrasound waves are emitted towards target location 202. In this arrangement, the ultrasound devices are said to be “mechanically steered”.

According to an aspect of the present application, a substrate 100 may be flexed to conform to a curved surface. When substrate 100 is placed in contact with and/or conforms to a curved surface, different support portions 102 may have different orientations relative to one another. Accordingly, different ultrasound devices 104 may have different orientations relative to one another, and as a result, may emit ultrasound waves in different directions (and/or receive ultrasound waves coming from different directions). One example of this arrangement is depicted in FIG. 2B, which illustrates a substrate 100 conforming to surface 200. Surface 200 may represent the surface of a human body.

One example of a servomechanism is a pneumatic actuator. A pneumatic actuator may be controlled for example using compressed air. In the example of FIG. 2C, bladders 112 made of rubber or other elastic materials may be disposed in contact with two or more support portions 102. The bladders 112 may be hollow and may have an inlet for receiving compressed air. The bladder may serve as a pneumatic actuator. That is, when compressed air is received within the hollow region, the bladder may expand thus exercising pressure on the corresponding support portions 102. As a result, the support portions may pivot or otherwise move relative to each other. The extent to which the support portions pivot or otherwise move relative to each other may be controlled by adjusting the pressure of the compressed air filling the bladders 112. It should be appreciated that, in other embodiments, a bladder 112 may serve as hydraulic actuators through the injection of fluid in the hollow region.

Another example of a servomechanism is a hydraulic actuator. A hydraulic actuator may be controlled using a fluid. An example of a hydraulic actuator is illustrated in FIG. 2D. In this example, actuators 162 are positioned in contact with two or more support portions 102. The actuators may include inlets 164 for receiving therein a fluid. The amount and/or pressure of the fluid flowing in the actuators 162 may determine the extent to which the actuators cause motion of the support portions 102 relative to each other. In some embodiments, the same fluid used for controlling the hydraulic actuator(s) may be used for cooling (e.g., for cooling ultrasound elements or other electronic components). As further illustrated in FIG. 2D, the support portions 102 may include inlets 166 for receiving therein the fluid (the same fluid used for the actuators or a different fluid). Flow of the fluid in a support portion 102 may cool the circuitry (e.g., ultrasound device 104) disposed on the support portion.

The system of FIG. 2E may be used to control the amount and/or pressure of the fluid conveyed to the actuators and the support portions for cooling. A fluid tank 140 may contain fluid therein. Fluid tank 140 may be in communication, via one or more fluid channels, to pump 142. Pump 142 may control the flow of the fluid conveyed for cooling purposes. Pump 144 may be used to control the flow of the fluid conveyed for actuating purposes. Pump 144 may be coupled to a controller configured to control the pump's operations. Pump 144 may be coupled to fluid tank 140 via pump 142 (as illustrated in FIG. 2E), directly, or in any other suitable arrangement. It should be appreciated that actuators 162 may be controlled via compressed air, rather than fluid, in some embodiments. In addition to, or in alternative to liquid cooling, in some embodiments, passive cooling may be used. For example, one or more support portions may be place in contact with heat sinks (e.g., copper heat sinks).

A specific example of a hydraulic actuator is illustrated in FIG. 2F, according to some non-limiting embodiments. Actuator 162 includes a fluid tank 170, an inlet 164 and a spring-loaded arm 172. When the fluid enters the tank 170 through inlet 164, the fluid exercises pressure on the arm 172, thus causing the arm to extend away from the tank. The presence of the spring ensures that the positon of the arm is restored when the fluid is removed from the tank.

In some embodiments, micro-channels configured to support flow of water or other fluids may be formed in the support portions 102, or in the substrates on which ultrasound elements 104 are fabricated, to improve cooling. One such channel may have a width that is between 10 μm and 100 m, such as between 40 μm and 60 μm, and a depth that is between 100 μm and 400 m, between 200 μm and 300 m. In one example, a 10 mm-long micro-channel may be formed on the silicon substrate hosting an ultrasound element 104. Water pressure at 60 psi may be allowed to flow through the micro-channel, and may allow cooling in excess of 1 KW/cm².

The servomechanism(s) may be adjusted for example to ensure that substrate 100 conforms to a desired surface, such as a portion of a human body. In some embodiments, the servomechanism(s) may be adjusted to arrange the support portions according to a desired configuration (e.g., a portion of an imaginary sphere, as will be described further below). In another example, a liquid-absorbing material may be used instead of (or in addition to) servomechanisms. The liquid absorbing material may be coupled to a coupling material whose ability to couple support portions may depend on the amount of liquid received from the liquid-absorbing material. The rate at which the liquid is provided to the coupling material may be controlled using for example a bottleneck-shaped channel or any suitable tapered shape.

In some embodiments, the servomechanisms may be adjusted in real-time, for example to ensure that the ultrasound signals are emitted in a desired direction throughout the duration of an operation and/or to ensure that substrate 100 conforms to a curved surface even if the geometry of the curved surface varies over time. In some embodiments, information indicative of the relative position of the support portions 102 may be obtained using joint sensors. The joint-sensors may sense forces, accelerations, torques, and/or motion. The joint sensors may provide real-time feedback on whether, for example, substrate 100 is arranged in such way to conform to a desired surface. Of course, other type of sensors other than joint sensors may be used including, but not limited to, ultrasound imaging sensors, accelerometers, gyroscopes, lasers, radars, cameras, Schlieren ultrasound beam imagers, hydrophones, EM trackers, and/or encoders.

In some embodiments, support portions 102 may be arranged relative to one another such that they form an imaginary sphere. In this way, mechanical alignment may be accomplished such that the ultrasound waves are focused on a common region (e.g., the center of the sphere). Of course, not all embodiments need to be arranged to form imaginary spheres. The arrangement of substrate 100 may be set prior to use and/or in real-time.

In some embodiments, matching fluids may be used to facilitate propagation of ultrasound waves from the ultrasound devices 104 to the human body. In one example, a bag containing water of other types of fluid may be positioned between the ultrasound devices 104 and the human body. The bag may have rigid walls or flexible outer walls. In some embodiments, individual bags are positioned between each support portion 102 and the human body. In other embodiments, one bag may be used for multiple support portions, such as for some or all the support portions.

In some embodiments, the frequency of the ultrasound waves may be chosen according to different considerations, such as the location and/or depth of the target region and/or the type of tissue being targeted. For example, since higher frequencies have, at least in some embodiments, larger focusing gains, more pressure can be generated in a tissue for the same intensity. However, ultrasound waves with higher frequencies suffer from increased attenuation loss as they propagate through a medium. As such, in some embodiments, trade-off considerations may be taken into account in choosing the frequency of the ultrasound waves. In some embodiments, the frequency may be chosen in the 0.1 MHz-3 MHz range or in the 1 MHz-3 MHz range. In some embodiments, the frequency used for HIFU may be larger than the frequency used for imaging. Lower frequencies in HIFU may ensure low attenuation as the ultrasound wave penetrates through the body. Higher frequencies for imaging may provide higher imaging resolutions. In one example, the frequency used for HIFU is between 0.1 MHz and 1 MHz and the frequency used for imaging is between 1 MHz and 3 MHz.

In some embodiments, the intensity generated by combining multiple ultrasound waves may be sufficiently large to cause a change in the acoustic properties of the medium. As a result, in some embodiments, focusing of the ultrasound waves may be distorted. Accordingly, in some embodiments, calibration of the ultrasound waves may be performed. The calibration may be performed periodically, or just prior to a medical procedure. Examples of calibration procedures are described further below.

In some embodiments, the direction of emission of the ultrasound devices may be controlled via “electronic steering.” Electronic steering may be achieved, at least in some embodiments, by controlling the phases of the signals with which different ultrasound elements are driven. As such, the ultrasound elements may be arranged to form a phased array. Electronic steering may be used for example in HIFU applications to orient the ultrasound beam produced by an ultrasound device towards a target region. In some embodiments, electronic steering may be used in combination with mechanical steering. For example, mechanical steering may be used to generally direct the emitted ultrasound beam to the target region, and electronic steering may be used for fine adjustments.

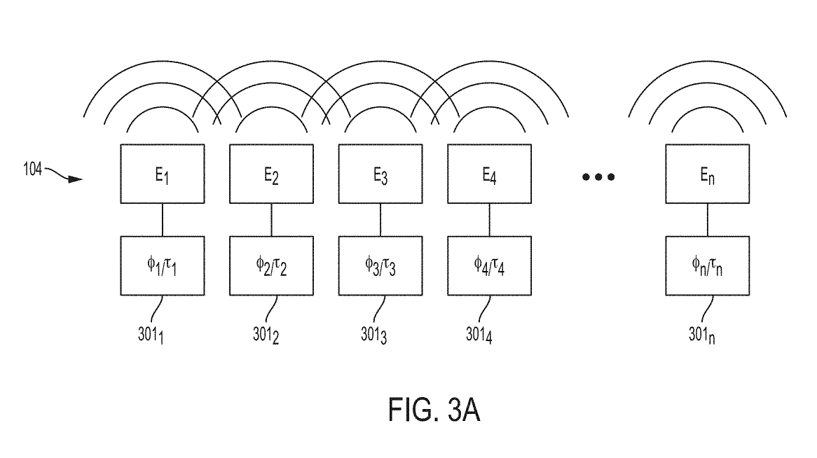

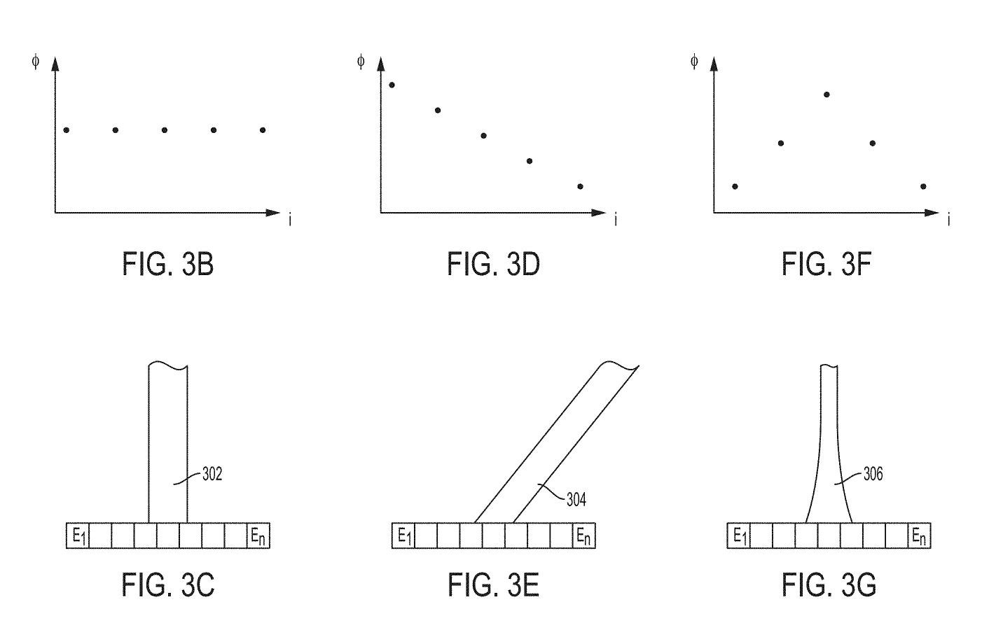

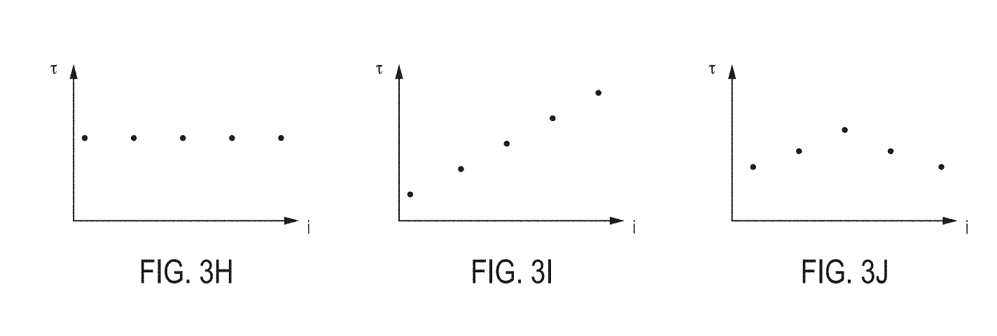

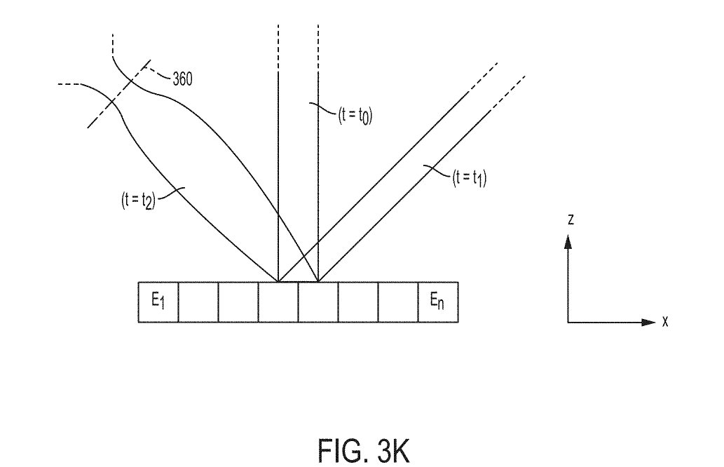

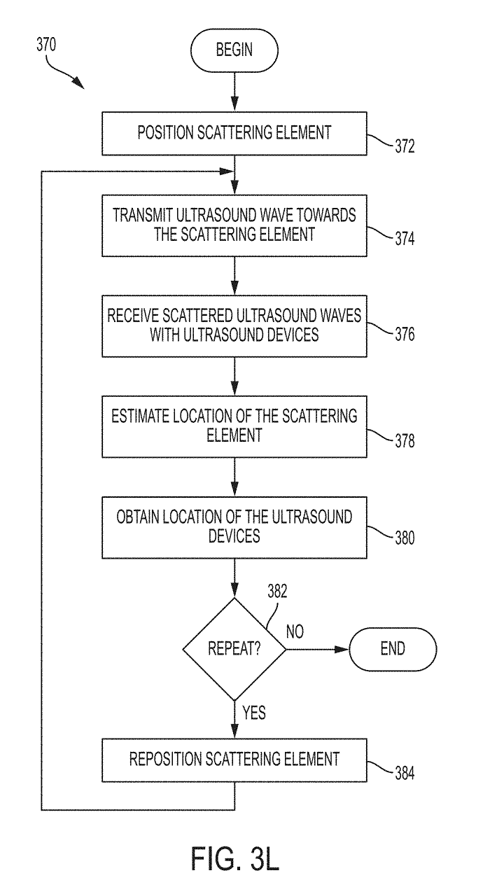

A representative phased array is depicted in FIG. 3A. In this configuration, an ultrasound device 104 comprises a plurality of ultrasound elements E₁, E₂, E₃, E₄ . . . E_{N}, where N may be greater than 10, greater than 100, greater than 1000, greater than 10000, or greater than 100000. The ultrasound elements may be arranged as a two-dimensional array, as a one-dimensional array, or may be sparsely arranged. Each ultrasound element may be configured to receive a drive signal having a certain phase and a certain time delay. For example, ultrasound element E₁ is driven by a signal having a phase Φ₁ and a delay τ₁, ultrasound element E₂ is driven by a signal having a phase Φ₂and a delay τ₂, ultrasound element E₃ is driven by a signal having a phase Φ₃ and a delay τ₃, ultrasound element E₄ is driven by a signal having a phase Φ₄ and a delay τ₄, and ultrasound element E_{N} is driven by a signal having a phase Φ_{N} and a delay τ_{N}. The phase and the delay of the drive signals may be controlled using signal drivers 301₁, 301₂, 301₃, 301₄, and 301_{N}. The signal drivers may comprise phase shifters and/or adjustable time delay units. According to the manner in which the various phases are controlled relative to one another, the individual ultrasound waves emitted by the ultrasound elements may experience different degrees of interference (e.g., constructive interference, destructive interference, or any suitable value in between). In some embodiments, the timing at which the ultrasound elements emit ultrasound signals may be adjusted relative to one another. This may be performed for example to ensure that the produced pulses (in the embodiments in which pulses are used) reach the target region simultaneously, thereby obtaining a desired intensity. Pulses of ultrasound waves may be used rather than continuous waves (CW) in different settings, including for example in micro-cavitation.

In some embodiments, the phases Φ₁, Φ₂, Φ₃, Φ₄ . . . Φ_{N} and/or time delays τ₁, τ₂, τ₃, τ₄ . . . τ_{N} may be controlled to cause the ultrasound waves to interfere with one another so that the resulting waves add together to increase the acoustic beam in a desired direction. The phases Φ₁, Φ₂, Φ₃, Φ₄ . . . Φ_{N} and/or time delays τ₁, τ₂, τ₃, τ₄ . . . τ_{N} may be controlled with respective signal drivers, which may be implemented for example using transistors and/or diodes arranged in a suitable configuration. In at least some of the embodiments in which the ultrasound elements are disposed on a semiconductor substrate, the signal drivers may be disposed on the same semiconductor substrate.

Examples of phase relationships and the beams resulting therefrom are depicted in FIGS. 3B-3G. FIG. 3B is a plot illustrating the phase of the signal with which each ultrasound element Eᵢ (i=1, 2 . . . N) is driven. In this example, the ultrasound elements are driven with uniform phases. As a result, the waves add together so that the acoustic beam 302 is mainly directed along the perpendicular to the plane of the ultrasound device (FIG. 3C).

In the example of FIG. 3D, the ultrasound elements are driven with phases arranged according to a linear relationship. As a result, the waves add together such that the acoustic beam 304 is angularly offset relative to the perpendicular to the plane of the ultrasound device (FIG. 3E).

In the example of FIG. 3F, the ultrasound elements are driven with phases arranged according to a quadratic relationship. As a result, the waves add together such that the acoustic beam 306 converges (FIG. 3G).

Of course, the phase relationship need not be linear or quadratic, as any other suitable phase relationship may be applied to the ultrasound elements. In some embodiments, multiple quadratic relationships may be used to generate multiple regions of highly focused energy simultaneously. In some embodiments, different time delays and/or different phases may be applied for different axes. In some embodiments, phases and/or the time delays may be adjusted to produce steering within a 3D field-of-view. This may be accomplished for example by adjusting azimuth and elevation of emission. In some embodiments, a portion of an ultrasound element may be occluded and may remain inactive. In some embodiments, HIFU elements may be configured to receive ultrasound signals and to identify potential occlusions and/or regions to be avoided (e.g., bones with strong returns or vital organs). In some embodiments, the signal drivers may be coupled to a controller (e.g., a digital circuit such a processor), which may be configured to vary the direction of emission over time, thus enabling ultrasound wave scanning. Ultrasound wave scanning may be used in imaging and/or in HIFU.

In some embodiments, the time delays τ₂, τ₃, τ₄ . . . τ_{N} may be controlled to ensure that the emitted beams reach the target region simultaneously and/or to ensure that the beams interfere in a substantially constructive fashion. The time delays may be adjusted in any suitable way. Examples of time delays associated with the ultrasound elements Eᵢ (i=1, 2 . . . N) are illustrated in FIGS. 3H-3J, according to some non-limiting embodiments. In the example of FIG. 3H, the time delays are uniform across the ultrasound elements. As a result, the ultrasound elements emit simultaneously. In the example of FIG. 3I, the time delays exhibit a linear relationship with respect to the ultrasound elements. In FIG. 3J, the time delays exhibit a quadratic relationship with respect to the ultrasound elements. In some embodiments, the manner in which the relative time delays are performed may depend on the shape of the surface on which the ultrasound device is positioned. For example, regions of the ultrasound device that are closer to the target region may be controlled with a greater time delay relative to regions that are farther away from the target region.

FIG. 3K is diagram of a non-limiting example illustrating how a phased array of the types described herein can be controlled to steer an acoustic beam. Initially, at time t=t₀, the phased array is controlled to direct the acoustic beam in a direction parallel the z-axis. At t=t₁, the phased array is controlled to redirect the acoustic beam at an angle relative to the z-axis. At t=t₂, the phased array is controlled to redirect the acoustic beam at another angle relative to the z-axis. In addition, at t=t₂, the phased array is controlled to cause a change in the focal length of the emitted beam. As illustrated, at t=t₂, the focus of the acoustic beam occurs at the plane 360.

In some embodiments, a calibration procedure may be used to ensure that the beams emitted by different ultrasound devices (whether configured for HIFU or imaging) are focused on the target region. Accordingly, some calibration procedures may be employed to determine the position of probes relative to the target region and/or the position of the probes relative to each other.

In some embodiments, a calibration procedure may be performed using a scattering element, such as small sphere of liquid or gel. Of course, not all scattering elements are limited to spheres as other shapes may be used. Scattering ultrasound signals from the scattering element may be used to determine suitable positions for the ultrasound devices. One example of a calibration procedure using a scattering element is illustrated in the flow chart of FIG. 3L. Calibration procedure 370 begins at act 372, in which a scattering element is positioned such that one or more ultrasound devices of the types described herein are generally oriented towards the scattering element. At act 374, an ultrasound wave is transmitted, by one or more of the ultrasound devices, towards the scattering element. The emitted ultrasound may be scattered (e.g., reflected) by the scattering element. The scattered ultrasound may be received by the ultrasound devices at act 376. At act 378, the location of the scattering element may be estimated based on the received scattered waves. In some embodiments, the location of the scattering element is estimated with respect to a plurality of local coordinate systems, where each local coordinate system represents is defined independently. For example, each local coordinate system may be centered at the location of a respective ultrasound device. Estimation of the location of the scattering element may be performed using a numerical solver, such as a least square method. At act 380, the locations of the ultrasound devices may be obtained. In some embodiments, such locations are obtained in a global coordinate system, that is, a single coordinate system shared among all the ultrasound devices. Such locations may be obtained by overlapping the locations of the scattering element estimated at act 378 in the global coordinate system. At act 382, it may be determined whether an additional iteration is appropriate. For example, it may be determined whether the estimated coordinates are sufficiently accurate. If it is determined that an additional iteration is appropriate, the scattering element (or a different scattering element) may be repositioned, and calibration procedure 370 continues to act 374. If it is determined that an additional iteration is not appropriate, calibration procedure 370 may end. In some embodiments, the position of the ultrasound devices may be adjusted based on the calibration procedure. For example, the position of multiple ultrasound devices may be adjusted using a suitable mechanism. Such a suitable mechanism may be arranged to adjust the position of at least one of the multiple ultrasound devices.

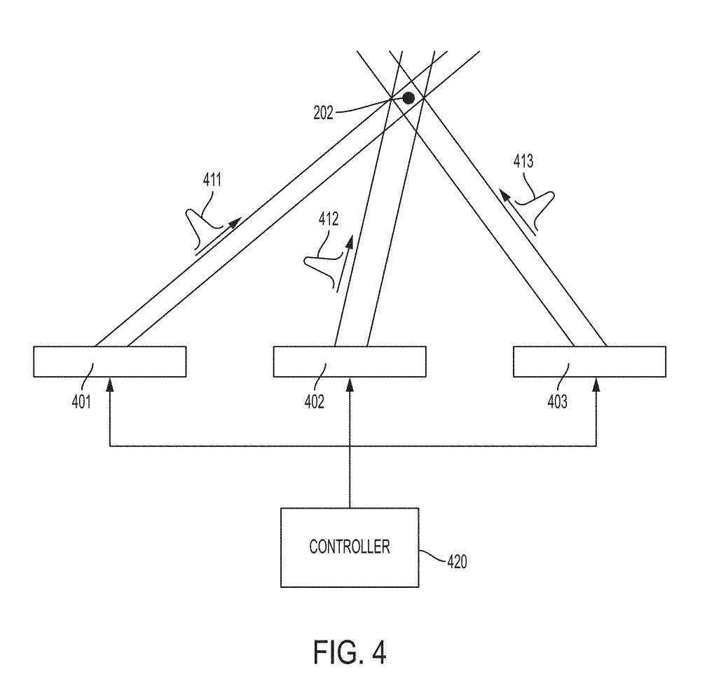

In some embodiments, multiple ultrasound devices may be used to concentrate ultrasound energy in a target region, thus increasing the intensity of the resulting wave. Alternatively, or additionally, multiple ultrasound devices may be controlled to time the emission of respective ultrasound pulses in a desired manner. One such configuration is depicted in FIG. 4. In this example, ultrasound elements 401, 402, and 403 are arranged as phased arrays, and are electronically controlled to direct the ultrasound beams towards target region 202. In this example, ultrasound elements 401, 402, 403 may be coupled to a controller 420 (e.g., a computer, a portable device or a processor, among others), which may be configured to adjust the relative phases and/or the relative timing of the emitted ultrasound signals. The phases and/or timing may be adjusted, for example, to ensure that the emitted ultrasound signals interfere substantially in-phase with one another at the target region 202. When the emitted ultrasound signals interfere substantially in-phase, constructive interference may be produced. By contrast, when the emitted ultrasound signals do not interfere substantially in-phase, destructive interference may be produced. When constructive, the interference may produce a larger acoustic intensity at the target region relative to the case in which the interference is destructive, thus making the procedure more effective. Timing may be adjusted in some embodiments so that the pulses 411, 412 and 413 reach the target region 202 simultaneously (or at least with some overlap in time). In this way, short pulses of large intensities may be obtained.

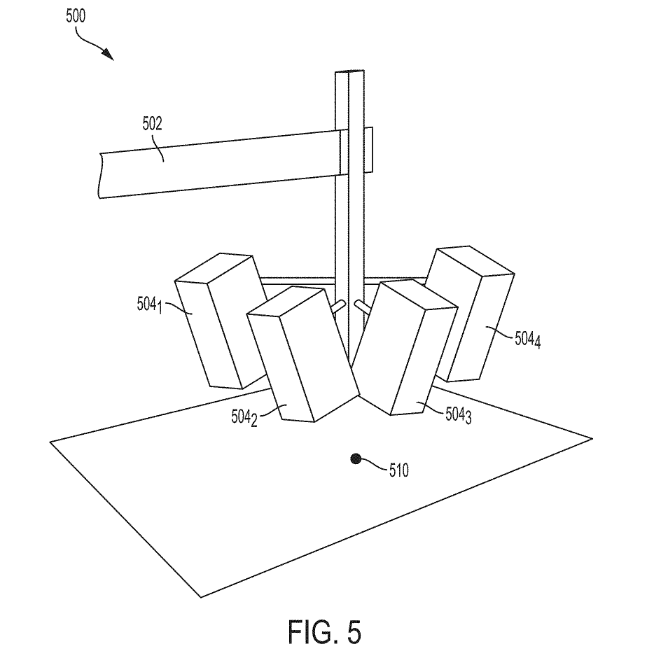

FIG. 5 illustrates a non-limiting example of a system for performing HIFU according to some aspects of the present application. As illustrated, system 500 comprises a support structure 502 and a plurality of probes including probes 504₁, 504₂, 504₃, and 504₄. Of course, system 500 is not limited to the specific number of probes shown in FIG. 5, as any other suitable number of probes may be used. For example, nine probes may be included, although some are not visible in FIG. 5 due to their positioning. The probes may comprise respective ultrasound devices arranged as imagers and/or HIFU elements. Support structure 502 may be configured to support the probes, and in some embodiments may be arranged to allow for the probe to be positioned independently from one another. For example, support structure 502 may comprise a plurality of hinges (or other types of couplers) for coupling the probes together. In this way, the probes can be oriented separately by actuating the respective hinges or other couplers.

In some embodiments, the probes may be oriented such that the HIFU beams emitted by the respective ultrasound devices are focused on a common region (e.g., a point) 510. In this way, the intensity of the resulting beam may be increased to a level suitable for HIFU.

In some embodiments, the ultrasound devices of the probes of system 500 may comprise phased arrays. As such, the HIFU beams emitted by the ultrasound devices may be electronically steered, thus improving the user's ability to focus beams on a desired location.

What is claimed is:

1. An apparatus comprising:
a substrate having a plurality of support portions including a first support portion and a second support portion, wherein the first support portion is connected to the second support portion through a coupler;
a first plurality of ultrasound elements configured as high-intensity focused ultrasound (HIFU) elements and disposed on the first support portion of the substrate; and
a second plurality of ultrasound elements configured as HIFU elements and disposed on the second support portion of the substrate.

2. The apparatus of claim 1, wherein the first and second plurality of ultrasound elements comprise capacitive micromachined ultrasound transducers (CMUT).

3. The apparatus of claim 1, wherein the coupler is selected from the group consisting of a hinge, a spring, a flexure, a beam, a joint and a sphere.

4. The apparatus of claim 1, further comprising a third plurality of ultrasound elements configured to receive ultrasound signals and disposed on the first support portion of the substrate.

5. The apparatus of claim 4, wherein the third plurality of ultrasound elements are configured as ultrasound imaging elements.

6. The apparatus of claim 1, further comprising an actuator coupled to the substrate, the actuator being configured to move the first support portion relative to the second support portion.

7. The apparatus of claim 6, wherein the actuator is selected from the group consisting of a pneumatic actuator, a hydraulic actuator and a servomotor.

8. A high intensity focused ultrasound (HIFU) apparatus, comprising:
a plurality of HIFU ultrasound-on-a-chip probes configured to emit electronically steerable beams, the plurality of HIFU ultrasound-on-a-chip probes being coupled to a support.

9. The HIFU apparatus of claim 8, further comprising a controller coupled to the plurality of HIFU ultrasound-on-a-chip probes and configured to control beam steering of the plurality of HIFU ultrasound-on-a-chip probes by adjusting relative phases of the electronically steerable beams.

10. The HIFU apparatus of claim 9, wherein the controller is configured to control beam steering of the plurality of HIFU ultrasound-on-a-chip probes by controlling a direction of emission and/or a focal length.

11. The HIFU apparatus of claim 8, wherein a first HIFU ultrasound-on-a-chip probe of the plurality of HIFU ultrasound-on-a-chip probes comprises an arrangement of capacitive micromachined ultrasound transducers (CMUTs) configured to provide HIFU.

12. The HIFU apparatus of claim 8, wherein the support comprises a plurality of support portions, the support portions being mechanically movable relative to each other.

13. The HIFU apparatus of claim 8, wherein at least one of the plurality of HIFU ultrasound-on-a-chip probes is configured to emit an acoustic intensity that is between 500 W/cm² and 20 KW/cm².

14. A method, comprising:
emitting, using an high-intensity focused ultrasound (HIFU) apparatus, at least one ultrasound signal towards at least one target region; and
adjusting, based on the emitting, direction of the at least one ultrasound signal via electronic steering.

15. The method of claim 14, wherein the at least one ultrasound signal is generated using at least one selected from the group consisting of: a capacitive micromachined ultrasound transducer (CMUT), piezoelectric transducer, lead zirconate titanate (PZT) element, lead magnesium niobate-lead titanate (PMN-PT) element, polyvinylidene difluoride (PVDF) element, high power ceramic element, and a PZT-4 ceramic element.

16. The method of claim 14, wherein the at least one ultrasound signal includes a high-intensity focused ultrasound (HIFU) signal and/or a non-HIFU ultrasound signal.

17. The method of claim 14, further comprising adjusting, based on the emitting, direction of the at least one ultrasound signal via mechanical steering.

18. The method of claim 17, wherein adjusting direction of the at least one ultrasound signal via mechanical steering includes adjusting position coordinates of at least one ultrasound element emitting the at least one ultrasound signal in relation to the at least one target region.

19. The method of claim 14, wherein adjusting direction of the at least one ultrasound signal via electronic steering includes controlling a phase of the at least one ultrasound signal.

20. The method of claim 14, wherein adjusting direction of the at least one ultrasound signal via electronic steering includes controlling a time delay of the at least one ultrasound signal.
